(19) United States
(12) Patent Application Publication
(10) Pub. No.: US 2021/0038095 A1
(43) Pub. Date: Feb. 11, 2021

(54) WEARABLE SYSTEM AND METHOD FOR DETERMINING BLOOD PRESSURE

(71) Applicant: CSEM Centre Suisse d'Electronique et de Microtechnique SA - Recherche et Développement, Neuchâtel (CH)

(72) Inventors: Martin Proença, Sugiez (CH); Josep Sola i Caros, Corcelles (CH); Olivier Chételat, Cudrefin (CH); Jonas Racine, Vevey (CH)

(21) Appl. No.: 17/044,488

(22) PCT Filed: Apr. 11, 2018
(86) PCT No.: PCT/IB2018/052542

§ 371 Date: Oct. 1, 2020

(51) Int. Cl. A61B 5/021; A61B 5/0402; A61B 5/00; A61B 5/0215; A61B 5/022

(52) U.S. Cl. CPC A61B 5/02125; A61B 5/0402; A61B 5/6804; A61B 5/02416; A61B 5/7221; A61B 5/0215; A61B 5/02233; A61B 5/0006

(57) ABSTRACT

A wearable measuring system configured for determining a blood pressure of a user, including: a first measuring unit including a PPG sensor, a first voltage measuring electrode and a first current injecting electrode; and a second measuring unit including a second voltage measuring electrode and a second current injecting electrode. The first measuring unit is removably attachable to a user's body first location such that a PPG signal can be measured by a PPG sensor at the first location, and the second measuring unit is removably attachable to a user's body second location, such that an ECG and an ICG signal can be measured between the first and second locations. The wearable measuring system further includes a signal processing module configured for processing the measured ECG, ICG and PPG signals to determine a blood pressure value.

### FIELD

The present invention concerns a wearable measuring system configured for determining a blood pressure of a user and a method for determining a blood pressure using the wearable measuring system.

### BACKGROUND

A device for determining a blood pressure of a user is described in Solà, et. al., “Chest Pulse Wave Velocity: a Novel Approach to Assess Arterial Stiffness”, IEEE Transactions on Biomedical Engineering, 58, 215-223, 2010. The blood pressure is determined by measuring a pre-ejection period (PEP) of the user, measuring a pulse arrival time (PAT) of the user, and subtracting them to obtain a pulse transit time (PTT) value. The PTT value is then transformed into a pulse wave velocity value, or a blood pressure (BP) value. The transformation of the PTT value in the BP value requires a calibration step, which is user dependent. A good accuracy in the determination of the PEP value is obtained by using a phono-cardiogram.

In Solà, J. et. al., “Non-invasive and non-occlusive blood pressure estimation via a chest sensor”, IEEE Transactions on Biomedical Engineering, 60, 3505-3513, July 2013, a BP sensor is disclosed based on a combination of an ECG sensor at the thorax, an ICG sensor at the thorax (upper-right, and lower-left electrodes) and a multi-channel PPG sensor at the sternum (central chest).

Proper electrode positioning is crucial to ICG, as it determines which physiological elements lie in the way of the current, leading to different impedance values. Band-type electrodes worn around the torso and the neck are often used. Spot electrodes have also been used as a solution that is reliable, more comfortable (no choking sensation) and more adapted to long-term measurements. Several electrode configurations have been proposed in the literature, including: four electrodes located on the chest, with one pair situated around the base of the neck and the other at the level of the xiphisternal joint, either centred or slightly off-centred; split-up pairs with current injecting electrodes on the back along the spine and voltage measuring electrodes on the sternum; or eight-electrode configurations (Niccomo ICG system) with four electrodes at the neck and four electrodes at the level of the xiphisternal joint.

In Qu et al., “Motion Artifact from Spot and Band Electrodes During Impedance Cardiography”, IEEE Transactions on Biomedical Engineering, 33, 1029-1036, 1986, several electrode configurations and their susceptibility to noise and motion artefacts were compared. They concluded that improving signal-to-noise ratio can only be achieve by making the body segment between two voltage electrodes as short as possible, under the condition of obtaining maximal useful signal. They also remarked that the change of distance between two voltage electrodes and the change of distance between voltage and current electrodes are the main causes of noise. They thus advised focusing on the upper thorax to pick up heart-related events and concluded that the front-back electrode array is a good option. However, no consensus exists when it comes to electrode positioning.

### SUMMARY

According to the invention, a wearable measuring system configured for determining a blood pressure of a user, comprises:

a first measuring unit comprising a PPG sensor, a first voltage measuring electrode and a first current injecting electrode;

a second measuring unit comprising a second voltage measuring electrode and a second current injecting electrode;

a wearable support destined to be worn on the user's body, the wearable support comprising: a first engagement feature configured to encompass a first location on the user body, the first measuring unit being removably attachable to the first engagement feature, such that a PPG signal can be measured by the PPG sensor at the first location; and a second engagement feature configured to encompass a second location on the user body, the second measuring unit being removably attachable to the second engagement feature, such that an ECG signal can be measured by the first and second voltage measuring electrodes and an ICG signal can be measured by the first and second voltage measuring and the first and the first and second current injecting electrodes;

a signal processing module configured for processing the measured ECG signal, the measured ICG signal and the measured PPG signal to determine a pre-ejection period (PEP) value, a pulse arrival time (PAT) value, and determine a blood pressure (BP) value from the determined PEP value and PAT value;

wherein the first location is at the shoulder, halfway between the upper parts of the scapula (preferably, the superior border of the scapula) and the clavicle; and the second location is at the level or below the fifth intercostal space.

The present invention further pertains to a method for determining a blood pressure of a user, comprising:

attaching the first measuring unit at the first engagement feature and attaching the second measuring unit at the second engagement feature;

measuring PPG signals at the first location;

measuring ECG signals and ICG signals between the first and the second locations;

processing the ECG signals and the PPG signals in the signal processing module to determine a PAT value;

processing the measured ECG signals and the measured ICG signals in the signal processing module to determine a PEP value; and

determining a pulse transit time (PTT) value from the determined PEP value and the determined PAT value; and determining a blood pressure (BP) value from the determined PTT value.

Attaching the first measuring unit and the second measuring unit respectively to the first and second locations allows for providing a canonical and repeatable ICG waveform signal. The first and second location configuration is advantageous when the first and second engagement features are to be arranged on a wearable support, such as a textile garment or T-shirt.

In contrast to measuring devices where the PPG sensor is located at the sternum, the wearable measuring system disclosed herein provides PPG signals that are more robust to heart mechanical activity. The PPG sensor at the first location does capture much less of the heart movements. Applying an optimal pressure on the PPG sensor by the wearable support at the first location is also greatly facilitated compared to when the PPG is at the sternum (due to concavity of sternum).

### BRIEF DESCRIPTION OF THE DRAWINGS

The invention will be better understood with the aid of the description of an embodiment given by way of example and illustrated by the figures, in which:

FIG. 1 illustrates a wearable measuring system comprising a first and second measuring units and configured for determining a blood pressure of a user, according to an embodiment;

FIG. 2 shows a detailed representation of the first and second measuring units, according to an embodiment;

FIG. 3 reports the reliability of investigated electrode configurations as a function of the position of the first and second measuring units;

FIG. 4 represents a PPG sensor comprised in the first measuring unit, according to an embodiment; and

FIG. 5 schematically represents a method for determining a blood pressure using the wearable measuring system, according to an embodiment.

### DETAILED DESCRIPTION OF POSSIBLE EMBODIMENTS

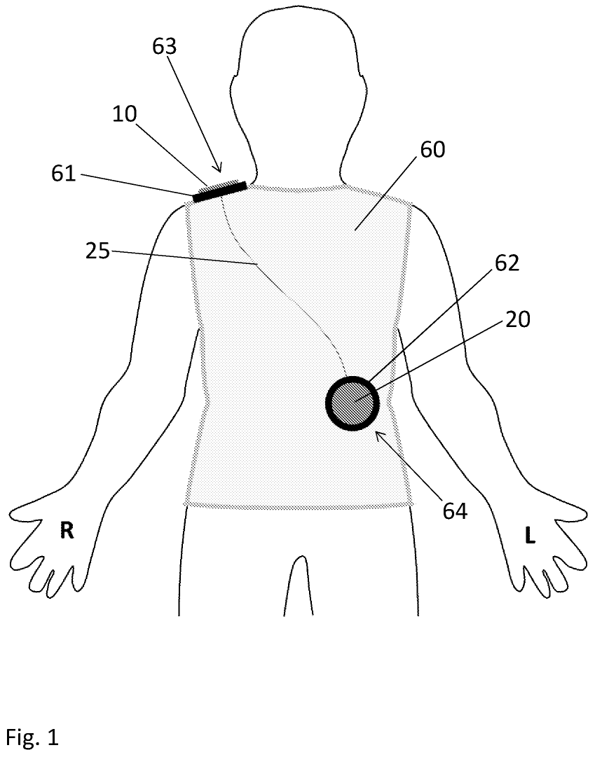

FIG. 1 illustrates a wearable measuring system configured for determining a blood pressure of a user, according to an embodiment. The wearable measuring system comprises a first measuring unit 10 destined to be placed at a first location 63 on the user body and a second measuring unit 20 destined to be placed at a second location 64 on the user body.

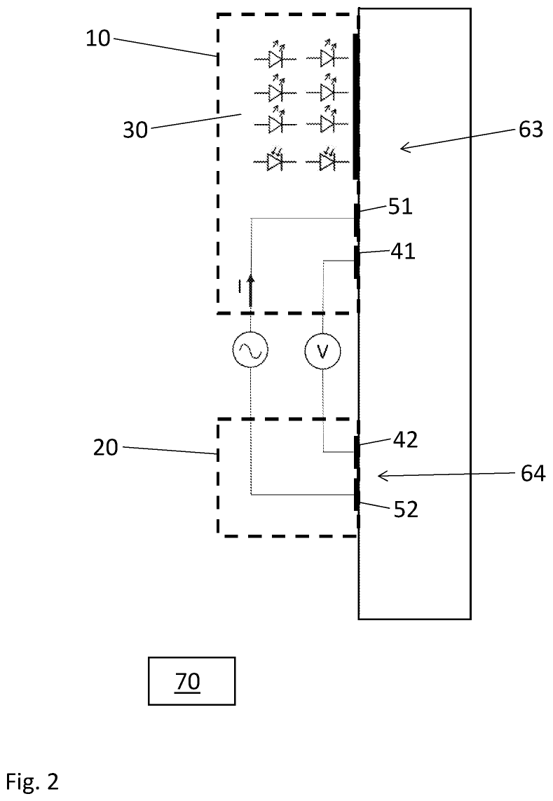

FIG. 2 shows a detailed representation of the first and second measuring units 10 and 20, according to an embodiment. The first measuring unit 10 comprises a PPG sensor 30, a first voltage measuring electrode 41 and a first current injecting electrode 51. The second measuring unit 20 comprises a second voltage measuring electrode 42 and a second current injecting electrode 52.

The wearable measuring system further comprises a wearable support 60 destined to be worn on the user's body. The wearable support 60 comprises a first engagement feature 61 configured to encompass a first location 63 on the user body, when the wearable support 60 is worn. The first engagement feature 61 is configured such that the first measuring unit 10 can be removably attachable to it. A PPG signal can then be measured by the PPG sensor 30 at the first location when the first measuring unit 10 is attached to the first engagement feature 61.

The wearable support 60 further comprises a second engagement feature 62 configured to encompass a second location 64 on the user body, when the wearable support 60 is worn. The second engagement feature 62 is configured such that the second measuring unit 20 can be removably attachable to it. Once both the first and second measuring units 10, 20 are attached to the first and second engagement features 61, 62 respectively, an ECG signal can be measured between the first and second locations 63, 64 by the first and second voltage measuring electrodes 41, 42. An ICG signal can also be measured between the first and second locations 63, 64 by the first and second voltage measuring 41, 42 and the first and second current injecting electrodes 51, 52.

The wearable measuring system further comprises a signal processing module 70 configured for processing the measured ECG signal, the measured ICG signal and the measured PPG signal. The signal processing module 70 is further configured for determining a pre-ejection period (PEP) value from the processed ECG and ICG signals; a pulse arrival time (PAT) value, from the processed ECG and PPG signals; and a pulse transit time (PTT) value from the determined PEP and PAT values. The signal processing module 70 can also be configure for determining a blood pressure (BP) value from the determined PEP value and PAT value.

According to a preferred embodiment, the first location 63 is at the shoulder, halfway between the upper parts of the scapula and the clavicle and the second location 64 is at the level or below the fifth intercostal space. Preferably, the upper parts of the scapula can be understood as the superior border of the scapula.

The reliability of several different four-electrode configurations were investigated. Here, four-electrode configuration means two voltage measuring electrodes and two current injecting electrode. These included configurations proposed by the literature (and variations thereof), such as positions including a measuring unit at the upper part of the chest or back, as suggested in US20060047214.

In addition, several other configurations were also investigated, all of which had to fulfil the constraint of keeping each current injecting electrode close to a voltage measuring electrode. Indeed, the current injecting electrodes and the voltage measuring electrodes are intended to be embedded in the same measurement unit (first and second measuring units 10, 20). The other configurations further had to fulfil the additional constraint of being easily embeddable in a wearable support (e.g. T-shirt). Electrodes at the neck being considered difficult to embed in such as support.

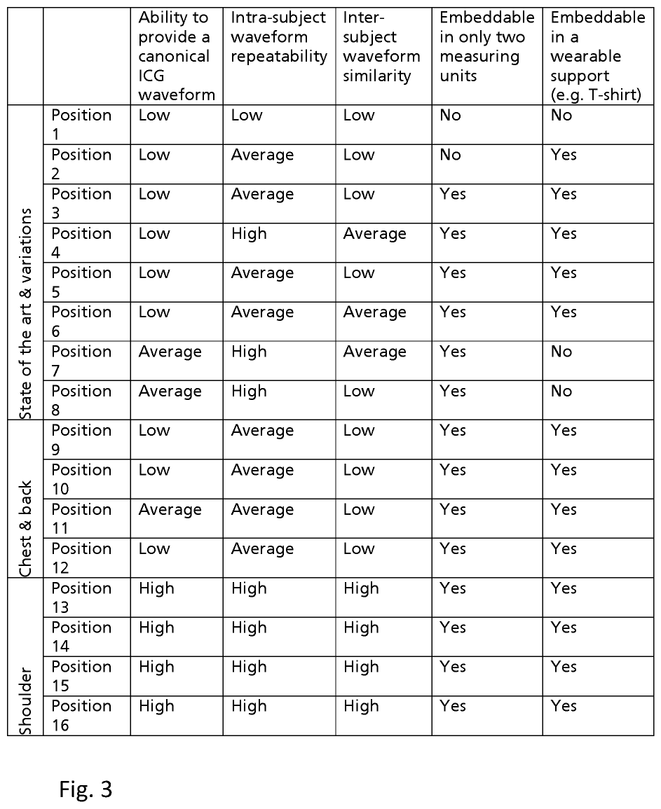

The reliability of each investigated electrode configuration was assessed in terms of its ability to measure a canonical form of the ICG waveform signal, as in Sherwood, et al., “Methodological Guidelines for Impedance Cardiography,” Psychophysiology, 27, 1-23, 1990, able to provide accurate PEP information, such as to obtain an accurate determined PEP value. The reliability of each investigated electrode configuration was further assessed by its ability of providing an ICG waveform signal with both low intra-subject variability (i.e. high similarity between individual ICG waveforms of a same subject) and low inter-subject variability (i.e. high similarity between average ICG waveforms of different subjects). The results of this investigations are summarized in FIG. 3.

The investigation findings indicate that the only positions of the four electrodes that provide a canonical and repeatable ICG waveform signal are those that include a measuring unit at the shoulder, combined with a measuring unit located at the level of the fifth intercostal space or lower. Although of particular interest given their ease of integration in a wearable support, configurations including electrode positions at the upper part of the chest or back, but not the shoulder, showed poor performance.

The first measuring unit 10 and the second measuring unit 20 can be connected by a single cable 25. The unique cable 25 can be a bundle of wires connecting the first measuring unit 10 and the second measuring unit 20. The wires can be used for synchronization and communication between both measuring units 10, 20. The cable 25 can be embedded or woven within the wearable support 60.

In a preferred embodiment, the first measuring unit 10 is connected to the second measuring unit 20 by a single wire, or the cable 25 comprises a single wire. The same wire is used for potential reference and/or for current return. No other wire connects the measuring units 10, 20. This one-wire approach results in simplified cabling and connectors.

In an embodiment, the wearable support 60 comprises one of a wearable textile support, a patch-like support, a belt or a garment.

In such configuration, instead of a physical wire, one can also connect the measuring units 10, 20 by contact directly to a conductive textile support, a patch-like support, belt or garment, etc.

The first and second engagement feature 61, 62 can comprise at least one of a snap, a pin, a magnet, a hook and loop fastener, a zipper, a press-fit, a snap-fit, a quick release fastener, or a torque limiting fastener.

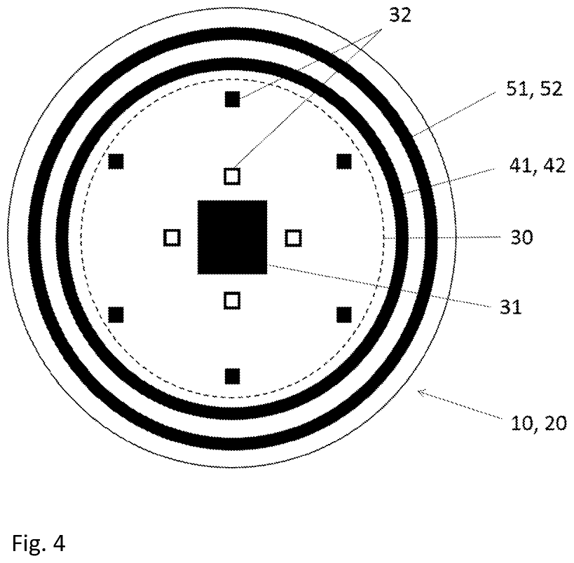

FIG. 4 represents a possible embodiment of the PPG sensor 30. In the illustrated example, the PPG sensor 30 comprises ten light emitters 32 arranged around a single photodetector 31. In such configuration, a PPG signal is measurable at the photodetector 31 for each light emitter 32.

The light emitters 32 can be positioned equidistant to the photodetector 31 or at different distances from the photodetector 31.

The light emitters 32 can emit with a unique wavelength or with a plurality of wavelengths. In the example of FIG. 4, the six light emitters 32 represented by the filled squares emits at a different wavelength than that of the four light emitters 32 represented by the empty squares.

Other configurations of the PPG sensor 30 are also contemplated. For example, the PPG sensor 30 can comprise a plurality of photodetectors 31.

In an embodiment, the signal processing module 70 is embedded in at least one of the first or second measuring units 10, 20. In this arrangement, the measured PPG, ICG and ECG signals processed in the processing module 70 and the PEP, PAT, PTT and PB values determined in the processing module 70 can be transmitted wirelessly to an external device (e.g. PC, tablet or smartphone).

In an alternative embodiment illustrated in FIG. 2, the signal processing module 70 is remote from the first and second measuring units 10, 20. The signal processing module 70 can then be communicatively connected wirelessly to the first and second measuring units 10, 20. In this arrangement, the measured PPG, ICG and ECG signals can be transmitted wirelessly from the first and the second measuring units 10, 20 to the processing module 70. The measured signals are then processed, and the PEP, PAT, PTT and PB values determined, in the remote signal processing module 70.

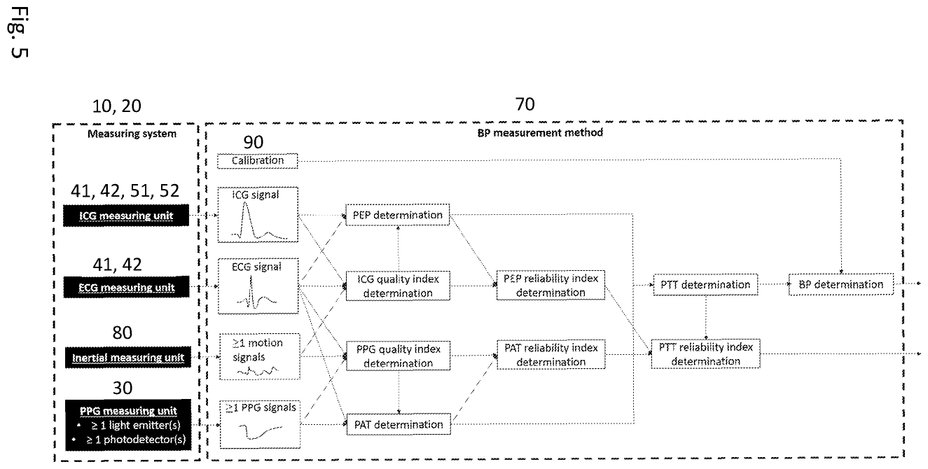

According to an embodiment schematically represented in FIG. 5, a method for determining a blood pressure of a user, comprises:

providing the wearable measuring system on the user's body;

attaching the first measuring unit 10 at the first engagement feature 61 and attaching the second measuring unit 20 at the second engagement feature 62;

once the first and second measuring units 10, 20 are attached, measuring PPG signals at the first location 63; and

measuring ECG signals and ICG signals between the first and second locations 63, 64.

The method further comprises the steps of:

processing the ECG signals and the PPG signals in the signal processing module 70 to determine a PAT value;

processing the measured ECG signals and the measured ICG signals in the signal processing module 70 to determine a PEP value;

determining a PTT value from the determined PEP value and the determined PAT value; and

determining a BP value from the determined PTT value.

In a variant embodiment, the method can further comprise the steps of:

processing the measured PPG signals in combination with the measured ECG signals to determine an PPG reliability index;

determining the PAT value using the PPG reliability index;

processing the measured ICG signals in combination with the measured ECG signals to determine an ICG reliability index; and

determining the PEP value using the ICG reliability index.

In another variant embodiment, the wearable measuring system further comprises an inertial measuring unit 80, such as a motion sensor delivering a motion signal representative of a motion of the user. In such arrangement, determining the PPG reliability index and determining the ICG reliability index can comprise using the motion signal.

In yet another variant embodiment, the method can further comprise the steps of:

determining a PAT reliability index by using the PPG reliability index;

determining a PEP reliability index by using the ICG reliability index; and

determining a PTT reliability index by using the PAT and PEP reliability indexes.

In yet another variant embodiment, the step of for determining the BP value from the determined PTT value can comprise using a user-dependent calibration 90. The user-dependent calibration 90 can be such as BP=f(V, P), where f is a function of the set V of variables and the set P of parameters. For instance, f can be a linear function, V can be a one-element set containing the PTT, and P contains the slope and the intercept of the linear function f.

The user-dependent calibration 90 can be based on a reference blood pressure measuring system. The reference blood pressure measuring system can comprise a brachial cuff or an invasive arterial line, or any other suitable blood pressure measuring system.

The user-dependent calibration can be based on anthropometric and physiological data of the user, such as weight, height, age, or gender.

### REFERENCE NUMBERS

10 first measuring unit
20 second measuring unit
25 cable
30 PPG sensor
31 photodetector
32 light emitter
41 first voltage measuring electrode
42 second voltage measuring electrode
51 first current injecting electrode
52 second current injecting electrode
60 wearable support
61 first engagement feature
62 second engagement feature
63 first location
64 second location
70 signal processing module
80 inertial measuring unit
90 calibration

What is claimed is:

1. A wearable measuring system configured for determining a blood pressure of a user, comprising:
a first measuring unit comprising a PPG sensor, a first voltage measuring electrode and a first current injecting electrode;
a second measuring unit comprising a second voltage measuring electrode and a second current injecting electrode;
a wearable support destined to be worn on the user's body, the wearable support comprising:
a first engagement feature configured to encompass a first location on the user body, the first measuring unit being removably attachable to the first engagement feature, such that a PPG signal can be measured by the PPG sensor at the first location; and
a second engagement feature configured to encompass a second location on the user body, the second measuring unit being removably attachable to the second engagement feature, such that an ECG signal can be measured by the first and second voltage measuring electrodes and an ICG signal can be measured by the first and second voltage measuring and the first and second current injecting electrodes;
a signal processing module configured for processing the measured ECG signal, the measured ICG signal and the measured PPG signal to determine a pre-ejection period (PEP) value, a pulse arrival time (PAT) value, and determine a blood pressure (BP) value from the determined PEP value and PAT value;
wherein the first location is at the shoulder, halfway between the upper parts of the scapula and the clavicle; and
the second location is at the level or below the fifth intercostal space.

2. The measuring system according to claim 1,
wherein the first measuring unit and the second measuring unit are connected by a single cable.

3. The measuring system according to claim 2,
wherein the cable comprises a single wire.

4. The measuring system according to claim 1,
wherein the wearable support comprises one of a wearable textile support, a patch-like support, a belt or a garment.

5. The measuring system according to claim 4,
wherein the first and second engagement feature comprises at least one of a snap, a pin, a magnet, a hook and loop fastener, a zipper, a press-fit, a snap-fit, a quick release fastener, or a torque limiting fastener.

6. The measuring system according to claim 1,
wherein the PPG sensor has a plurality of light emitters and at least one photodetector such that a PPG signal is measurable at the photodetector for each light emitter.

7. The measuring system according to claim 6,
wherein said plurality of light emitters emit with a plurality of wavelengths.

8. The measuring system according to claim 6 or 7,
wherein the light emitters are positioned at different distances from the at least one photodetector.

9. The measuring system according to claim 1,
wherein the signal processing module is embedded in at least one of the first or second measuring units.

10. The measuring system according to claim 9,
wherein the processed signals and determined values are transmitted wirelessly to an external device.

11. The measuring system according to claim 1,
wherein the signal processing module is remote from the first measuring unit and the second measuring unit is communicatively connected wirelessly to the first and second measuring units.

12. The measuring system of claim 11,
wherein the measured signals are transmitted wirelessly from the first measuring unit and the second measuring unit to the processing module; and
wherein the measured signals are processed in the remote signal processing module.

13. A method for determining a blood pressure of a user, comprising:
providing the wearable measuring system on the user's body, the wearable measuring system comprising a first measuring unit comprising a PPG sensor, a first voltage measuring electrode and a first current injecting electrode; a second measuring unit comprising a second voltage measuring electrode and a second current injecting electrode; a wearable support destined to be worn on the user's body, the wearable support comprising a first engagement feature configured to encompass a first location on the user body, the first measuring unit being removably attachable to the first engagement feature, such that a PPG signal can be measured by the PPG sensor at the first location; and a second engagement feature configured to encompass a second location on the user body, the second measuring unit being removably attachable to the second engagement feature, such that an ECG signal can be measured by the first and second voltage measuring electrodes and an ICG signal can be measured by the first and second voltage measuring and the first and second current injecting electrodes; a signal processing module configured for processing the measured ECG signal, the measured ICG signal and the measured PPG signal to determine a PEP value, a PAT value, and determine a BP value from the determined PEP value and PAT value; wherein the first location is at the shoulder, halfway between the upper parts of the scapula and the clavicle; and
the second location is at the level or below the fifth intercostal space;
attaching the first measuring unit at the first engagement feature and attaching the second measuring unit at the second engagement feature;
measuring PPG signals at the first location;
measuring ECG signals and ICG signals between the first and second locations;
processing the ECG signals and the PPG signals in the signal processing module to determine a PAT value;
processing the measured ECG signals and the measured ICG signals in the signal processing module to determine a PEP value;
determining a pulse transit time (PTT) value from the determined PEP value and the determined PAT value; and
determining a BP value from the determined PTT value.

14. The method according to claim 13,
further comprising the steps of:
processing the measured PPG signals in combination with the measured ECG signals to determine an PPG reliability index;
determining the PAT value using the PPG reliability index;
processing the measured ICG signals in combination with the measured ECG signals to determine an ICG reliability index; and
determining the PEP value using the ICG reliability index.

15. The method according to claim 14,
wherein the wearable measuring system further comprises a motion sensor delivering a motion signal representative of a motion of the user; and
wherein determining the PPG reliability index and determining the ICG reliability index comprise using the motion signal.

16. The method according to claim 13,
further comprising the steps of:
determining a PAT reliability index by using the PPG reliability index;
determining a PEP reliability index by using the ICG reliability index; and
determining a PTT reliability index by using the PAT and PEP reliability indexes.

17. The method according to claim 14,
further comprising using a user-dependent calibration for determining the BP value from the determined PTT value.

18. The method according to claim 17,
wherein the user-dependent calibration is based on a reference blood pressure measuring system.

19. The method according to claim 18,
wherein the reference blood pressure measuring system comprises a brachial cuff or an invasive arterial line.

20. The method according to claim 17,
wherein the user-dependent calibration is based on anthropometric and physiological data of the user.
